(19) United States
(12) Patent Application Publication
(10) Pub. No.: US 2025/0072758 A1
(43) Pub. Date: Mar. 6, 2025

(54) SYSTEMS, METHODS, AND DEVICES OF WEARABLE ELECTRO-ACOUSTIC MONITORING

(71) Applicant: GS-HealthMatrix, LLC, Los Angeles, CA (US)

(72) Inventors: Joseph Schwab, Los Angeles, CA (US); Amirhossein Yazdkhasti, Los Angeles, CA (US); Hamid Ghaednia, Los Angeles, CA (US)

(73) Assignee: GS-HealthMatrix, LLC, Los Angeles, CA (US)

(21) Appl. No.: 18/819,223

(22) Filed: Aug. 29, 2024

Related U.S. Application Data
(60) Provisional application No. 63/579,605, filed on Aug. 30, 2023.
(60) Provisional application No. 63/579,616, filed on Aug. 30, 2023.
(60) Provisional application No. 63/579,627, filed on Aug. 30, 2023.
(60) Provisional application No. 63/579,633, filed on Aug. 30, 2023.
(60) Provisional application No. 63/579,640, filed on Aug. 30, 2023.
(60) Provisional application No. 63/579,647, filed on Aug. 30, 2023.
(60) Provisional application No. 63/579,663, filed on Aug. 30, 2023.

(51) Int. Cl. A61B 5/00; A61B 5/0537; A61B 5/256; A61B 5/296; A61B 5/395; A61B 90/00

(52) U.S. Cl. CPC A61B 5/0093; A61B 5/0048; A61B 5/0537; A61B 5/256; A61B 5/296; A61B 5/395; A61B 5/4504; A61B 5/4523; A61B 5/6803; A61B 5/6806; A61B 90/06; A61B 2090/064; A61B 2560/0468; A61B 2562/0204; A61B 2562/0247; A61B 2562/066; A61B 2562/164

(57) ABSTRACT

Systems, methods, and devices include a wearable device to stimulate or analyze biological systems or implantable objects. The system includes a substrate material and a plurality of acoustic actuators disposed on the substrate material. The plurality of acoustic actuators are operable to generate an acoustic stimulation signal directed to a target area. The system also includes a plurality of acoustic sensors disposed on the substrate material and operable to receive an acoustic response signal from the target area. Furthermore, a plurality of electrical electrodes disposed on the substrate material are operable to generate an electrical stimulation signal directed to the target area and receive an electrical response from the target area. The substrate material forms a sleeve or a cuff, a glove, a head cap, a back harness, a waist binder, a torso binder, and/or an abdominal binder.

### CROSS-REFERENCE TO RELATED APPLICATIONS

This application claims priority to U.S. Provisional Application Ser. No. 63/579,605 filed Aug. 30, 2023 and titled “FRUSTRATED TOTAL INTERNAL REFLECTION (FTIR) SURFACE TOPOGRAPHY AND COMPOSITION ANALYSIS SYSTEMS, METHODS, AND DEVICES;” U.S. Provisional Application Ser. No. 63/579,616 filed Aug. 30, 2023 and titled “SYSTEMS, METHODS, AND DEVICES OF WEARABLE ELECTRO-ACOUSTIC MONITORING;” U.S. Provisional Application Ser. No. 63/579,627 filed Aug. 30, 2023 and titled “SYSTEMS, METHODS, AND DEVICES FOR ACOUSTICALLY ENHANCING IMPLANTS;” U.S. Provisional Application Ser. No. 63/579,633 filed Aug. 30, 2023 and titled SYSTEMS, METHODS, AND DEVICES WITH SENSORS HAVING MULTIPLE DETECTION SIGNAL TYPES;” U.S. Provisional Application Ser. No. 63/579,640 filed Aug. 30, 2023 and titled MULTI-DEVICE HEALTH PARAMETER MONITORING SYSTEMS, METHODS, AND DEVICES;” U.S. Provisional Application Ser. No. 63/579,647 filed Aug. 30, 2023 and titled FRUSTRATED TOTAL INTERNAL REFLECTION (FTIR)-BASED HEALTH PARAMETER DETECTION SYSTEMS, METHODS, AND DEVICES;” and U.S. Provisional Application Ser. No. 63/579,663 filed Aug. 30, 2023 and titled “SYSTEMS, METHODS, AND DEVICES FOR NEUROLOGICAL AND/OR MUSCOSKELETAL PARAMETER CHARACTERIZATION;” the entireties of which are herein incorporated by reference.

### BACKGROUND

Conventional electrical sensing and actuation systems do not provide accurate information about mechanical activity of the living body. For instance, these systems typically do not provide direct information about structure and physiology of biological systems, and their interaction with implantable materials.

It is with these observations in mind, among others, that various aspects of the present disclosure were conceived and developed.

### SUMMARY

The systems, methods, and devices disclosed herein can address the aforementioned issues. For instance, a wearable device to stimulate or analyze biological systems or implantable objects can include a substrate material; a plurality of acoustic actuators disposed on the substrate material and operable to generate an acoustic stimulation signal directed to a target area; a plurality of acoustic sensors disposed on the substrate material and operable to receive an acoustic response signal from the target area; and/or a plurality of electrical electrodes disposed on the substrate material and operable to generate an electrical stimulation signal directed to the target area and receive an electrical response from the target area.

In some examples, the substrate material can be a flexible materials including at least one of a fabric, a natural biological materials, a polymer, or a metal. Also, the substrate material can form a sleeve or a cuff, a glove, and/or a head cap. Furthermore, the substrate material can form a back harness, a waist binder, a torso binder, or an abdominal binder. Also, the plurality of acoustic actuators, the plurality of acoustic sensors, and the plurality of electrical electrodes can be positioned along a spinal region of the back harness, the waist binder, the torso binder, or the abdominal binder.

In some instances, a wearable device to stimulate or analyze biological systems or implantable objects can include a substrate material and/or a plurality of sensors and actuators disposed on the substrate material as an array. The plurality of sensors and actuators can be operable to generate an acoustic stimulation signal directed to a target area; generate an electrical stimulation signal directed to the target area; receive an acoustic response signal from the target area; and/or receive an electrical response signal from the target area.

In some scenarios, the plurality of sensors and actuators can be substantially evenly distributed on the substrate material. Additionally, the one of the plurality of sensors and actuators can include at least one of a piezoelectric transducer; a speaker and a microphone; and/or one or more metamaterials.

In some examples, a method to stimulate or analyze biological systems or implantable objects can include positioning a wearable device at a target area on a body of a user; generating, using one or more acoustic actuators disposed on the wearable device, an acoustic stimulation signal directed to the target area; generating, using one or more electrical electrodes on the wearable device, an electrical stimulation signal directed to the target area; receiving, using one or more acoustic sensors; an acoustic response signal from the target area; and/or receiving, using one or more electrical sensors, an electrical response signal from the target area.

In some instances, the method can include changing an electrical property of the target area with the acoustic stimulation signal, the electrical response signal representing the changing of the electrical property. The method can also include performing, with the acoustic stimulation signal, acoustic therapy or a quality assessment on at least one of muscle tissue, bone tissue, tendon tissue, or implant tissue. Additionally, the method can include performing, with the acoustic stimulation signal or the acoustic response signal, acoustic monitoring for biological systems, or implantable objects. Moreover, the method can include performing, with the electrical response signal, an electromyography (EMG) for biological systems, muscle tissue, tendon tissue, or a nerve activate.

In some scenarios, the method can include performing, with the electrical stimulation signal and the electrical response signal, an electrical impedance measurement or an electrical capacitance measurement for tissue of the target area. The electrical impedance measurement or the electrical capacitance measurement can indicate at least one of a volumetric change, a composition change, a health of a tissue or a biological system, a mechanical change, an electrical change, or a nerve activate. Additionally, the method can include performing, with the acoustic stimulation signal and the electrical response signal, an electro-acoustic stimulation in which the acoustic stimulation signal stimulates a nerve, and the nerve generates the electrical response signal. The method can also include performing, with the acoustic stimulation signal and the electrical response signal, an electro-acoustic muscle characterization in which the electrical response signal or the acoustic response signal is generated responsive to the acoustic stimulation signal and the electrical stimulation signal. A frequency or an intensity of the acoustic stimulation signal can correspond to a type of tissue, organ, or cells at the target area. Moreover, the frequency can be an ultrasonic frequency, and the type of tissue can include a tendon, a muscle, a bone, or cartilage receiving the acoustic stimulation signal for healing.

### BRIEF DESCRIPTION OF THE DRAWINGS

The foregoing summary, as well as the following detailed description, will be better understood when read in conjunction with the appended drawings. For the purpose of illustration, there is shown in the drawings certain embodiments of the disclosed subject matter. It should be understood, however, that the disclosed subject matter is not limited to the precise embodiments and features shown. The accompanying drawings, which are incorporated in and constitute a part of this specification, illustrate an implementation of systems and methods consistent with the disclosed subject matter and, together with the description, serves to explain advantages and principles consistent with the disclosed subject matter, in which:

FIG. 1 illustrates an example system including a wearable device for tissue stimulation and/or analysis.

FIGS. 2A-2F illustrate example diagrams of cross-domain analyses of a wearable device using electrical signals and/or acoustic signals.

FIGS. 3A and 3B illustrate an example wearable cuff and/or sleeve device for tissue stimulation and/or analysis.

FIGS. 4A and 4B illustrate an example back harness and/or spinal strip device for tissue stimulation and/or analysis.

FIGS. 5A and 5B illustrate an example glove and/or hand device for tissue stimulation and/or analysis.

FIGS. 6A and 6B illustrate an example head device for tissue stimulation and/or analysis.

FIGS. 7A and 7B illustrate an example waist or torso device for tissue stimulation and/or analysis.

FIGS. 8A-8D illustrate example wearable devices with non-human subjects for tissue stimulation and/or analysis.

FIG. 9 illustrates an example method of performing tissue stimulation or analysis with a wearable device.

### DETAILED DESCRIPTION

It will be appreciated that for simplicity and clarity of illustration, where appropriate, reference numerals have been repeated among the different figures to indicate corresponding or analogous elements. In addition, numerous specific details are set forth in order to provide a thorough understanding of the embodiments described herein. However, it will be understood by those of ordinary skill in the art that the embodiments described herein can be practiced without these specific details. In other instances, methods, procedures and components have not been described in detail so as not to obscure the related relevant feature being described. Also, the description is not to be considered as limiting the scope of the embodiments described herein. The drawings are not necessarily to scale and the proportions of certain parts may be exaggerated to better illustrate details and features of the present disclosure.

The phraseology and terminology employed herein are for the purpose of description and should not be regarded as limiting. For example, the use of a singular term, such as, “a” is not intended as limiting of the number of items. Also, the use of relational terms such as, but not limited to, “top,” “bottom,” “left,” “right,” “upper,” “lower,” “down,” “up,” and “side,” are used in the description for clarity in specific reference to the figures and are not intended to limit the scope of the presently disclosed technology or the appended claims. Further, it should be understood that any one of the features of the presently disclosed technology may be used separately or in combination with other features. Other systems, methods, features, and advantages of the presently disclosed technology will be, or become, apparent to one with skill in the art upon examination of the figures and the detailed description. It is intended that all such additional systems, methods, features, and advantages be included within this description, be within the scope of the presently disclosed technology, and be protected by the accompanying claims.

Further, as the presently disclosed technology is susceptible to embodiments of many different forms, it is intended that the present disclosure be considered as an example of the principles of the presently disclosed technology and not intended to limit the presently disclosed technology to the specific embodiments shown and described. Any one of the features of the presently disclosed technology may be used separately or in combination with any other feature. References to the terms “embodiment,” “example,” and/or the like in the description mean that the feature and/or features being referred to are included in, at least, one aspect of the description. Separate references to the terms “embodiment,” “examples,” and/or the like in the description do not necessarily refer to the same example and are also not mutually exclusive unless so stated and/or except as will be readily apparent to those skilled in the art from the description. For instance, a feature, structure, process, step, action, or the like described in one embodiment may also be included in other embodiments but is not necessarily included. Thus, the presently disclosed technology may include a variety of combinations and/or integrations of the examples described herein. Additionally, all aspects of the present disclosure, as described herein, are not essential for its practice. Likewise, other systems, methods, features, and advantages of the presently disclosed technology will be, or become, apparent to one with skill in the art upon examination of the figures and the description. It is intended that all such additional systems, methods, features, and advantages be included within this description, be within the scope of the presently disclosed technology, and be encompassed by the claims.

Any term of degree such as, but not limited to, “substantially,” as used in the description and the appended claims, should be understood to include an exact, or a similar, but not exact configuration. For example, “a substantially planar surface” means having an exact planar surface or a similar, but not exact planar surface. Similarly, the terms “about” or “approximately,” as used in the description and the appended claims, should be understood to include the recited values or a value that is three times greater or one third of the recited values. For example, about 3 mm includes all values from 1 mm to 9 mm, and approximately 50 degrees includes all values from 16.6 degrees to 150 degrees.

The term “coupled” is defined as connected, whether directly or indirectly through intervening components, and is not necessarily limited to physical connections. The connection can be such that the objects are permanently connected or releasably connected. The terms “comprising,” “including” and “having” are used interchangeably in this disclosure. The terms “comprising,” “including” and “having” mean to include, but not necessarily be limited to the things so described. The term “real-time” or “real time” means substantially instantaneously.

Lastly, the terms “or” and “and/or,” as used herein, are to be interpreted as inclusive or meaning any one or any combination. Therefore, “A, B, or C” or “A, B, and/or C” mean any of the following: “A,” “B,” or “C”; “A and B”; “A and C”; “B and C”; “A, B and C.” An exception to this definition will occur only when a combination of elements, functions, steps or acts are in some way inherently mutually exclusive.

The systems, methods, and devices disclosed herein include a wearable device to perform simultaneous electrical and acoustic stimulation and analysis of different living objects such as the human body. The system(s) can perform a cross-domain analysis between the electrical and acoustic properties of cells and tissue. Acoustic measurements and electrical measurements can be taken independently or dependently, such that the effects of acoustic signals on electrical signals, and vice versa, can be measured. For example, acoustic stimulation of cells and/or tissue can manipulate their electrical properties to enable specific measurements and improved understanding of their biomechanical properties.

The wearable devices disclosed herein are capable of independent acoustic and electrical sensing and actuating simultaneously. At the same time, these wearable can be used to stimulate the body in one of the electrical domain or the acoustic domain and monitor the reaction of the body using the other domain. The device can be used for different procedures such as an acoustic stimulation procedure; an acoustic diagnostics and/or monitoring procedure; an electrical mythography procedure; an electrical impedance tomography procedure; an electro-acoustic neuromodulation procedure; an electro-acoustic muscle characterization procedure; and/or combinations thereof.

Accordingly, the wearable device disclosed herein can have a variety of uses for different target regions of the user. The wearable device can be used to monitor muscle activity, muscle health, muscle volume, muscle strength, and/or a muscle healing process. Additionally, the systems disclosed herein can be used to monitor bone density, water content, and/or a bone healing process. The system can focus acoustic energy at a desired point to improve a healing process, manipulate or damage cancer cells, and/or excite nerves. Also, the system can be used to monitor an implant healing process and/or perform a diagnosis of a defect in implant fixation. Additionally, or alternatively, the system can be used to monitor a tendon healing process and/or diagnose a defect in the tendon. Furthermore, the system can be used to improve cell attachment to foreign objects in the human body including implants grafts, fracture plates, screws, and/or other fixations.

Additional advantages of the systems, methods, and devices discussed herein will become apparent from the detailed description below.

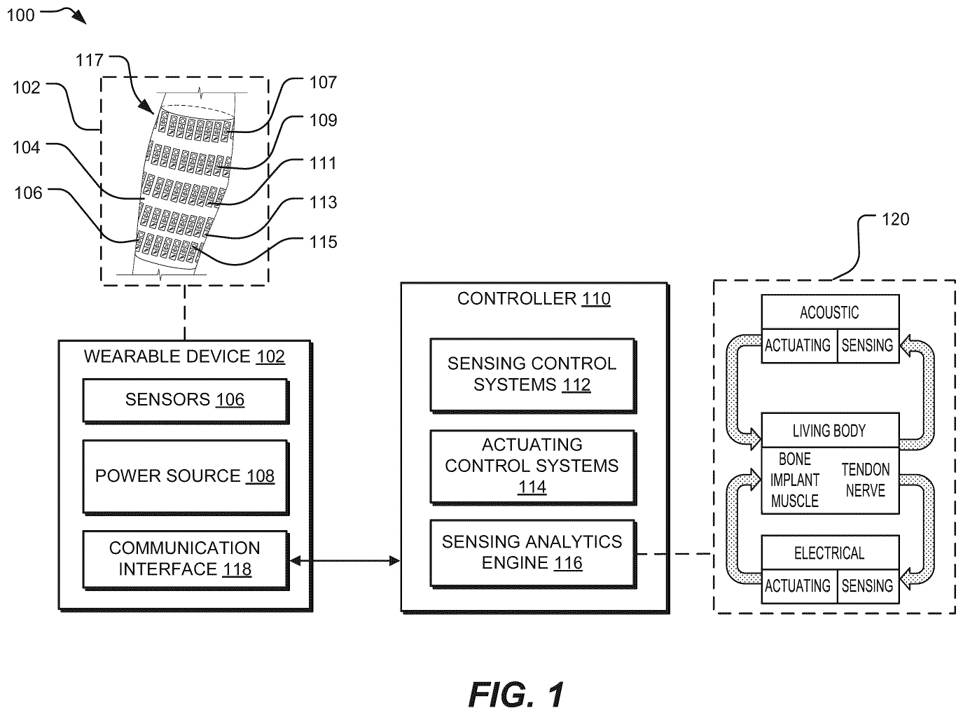

FIG. 1 illustrates an example system 100 including a wearable device 102, such as an acoustic and/or electric wearable device. The wearable device 102 can include a substrate body 104 formed of fabric, plastic, or other flexible or partially flexible materials and/or various other types of material to form different shapes, sizes, and form-factors. The wearable device 102 can also include one or more sensors and/or actuators, such as a plurality of sensors/actuators 106. The plurality of sensors/actuators 106 can include any combination of acoustic actuators 107, acoustic sensors 109, electrical actuators 111, and/or electrical sensors 113. The wearable device 102 can include one or more integrated sensors/actuators 115, the integrated sensor/actuator 115 being a combination of the acoustic sensor 109, the acoustic actuator 107, and/or the electrical electrode (e.g., the electric actuator 111 and/or the electric sensor 113). The wearable device 102 can include an array 117 of the integrated sensors. The wearable device 102 can also include a power source 108, such as a battery and/or an AC power adapter disposed on the substrate body 104 (or separate from the substrate body 104). The wearable device 102 can also include a controller 110, such as a processor or microcontroller, for implementing a sensing control system 112, an actuation control system 114, and/or a sensing analytics engine 116. The controller 110 and/or any components of the controller 110 can be integral with the wearable device 102 (e.g., disposed on the substrate body 104), or the controller 110 and/or any components of the controller 110 can be remote or separate from the substrate body 104. For instance, the substrate body 104 can include a communication interface 118 (e.g., a wireless communication interface) for communicating with the controller 110, for instance, to send data collected from the plurality of sensors/actuators 106 to the sensing analytics engine 116. The sensing analytics engine 116 can perform one or more sensor data analyses and/or cross-domain analyses 120 using the sensor data.

For instance, the acoustic-electric wearable device 102 can generate acoustic and electrical signal with different waveforms (e.g., pure tone, gaussian waves, or so forth), different frequencies (e.g., 1 Hz to 10 MHz) and/or different intensities from one or multiple actuators. The generated signals can interact with soft and hard tissues. This interaction may lead to transformation of tissues or transformation of a wave. The transformed wave can be measured using the same or different array 117 of electrical and acoustic sensors (e.g., the plurality of sensors/actuators 106). Furthermore, the wearable device 102 can be loosened or tightened while sensors and actuators are evenly distributed, and/or locations of the sensors/actuators can be recorded. The collection of sensor data may be transmitted to an external data base, mobile devices (e.g., a cell phone) and/or a data acquisition system through wired or wireless communication interfaces (e.g., communication interface 118). Furthermore, the plurality of sensors/actuators 106 can include one or more piezoelectric transducer used for momentous sensing and actuating, and/or one or more micro-electromechanical system (MEMS) microphones and/or speakers, which can be used for a high-density arrays. Furthermore, the sensing analytics engine 116 can include one or more machine learning (ML) models for extracting information from the measured signals (e.g., using various architectures to perform a time series analysis) or a combination of one or more signal processing methods and/or analytical and/or machine learning algorithms for signal processing, which, in some scenarios, forms part of a cross-domain analysis 120.

In some examples, the outputs of the sensors 106 can be particularized for different use case scenarios. Acoustic actuators 106 can output a particular frequency and/or a particular amplitude corresponding to a type of tissue being stimulated and/or analyzed. For instance, an ultrasound output frequency can correspond to a particular use-case of tendon healing. Other output frequencies can be used for targeting bone tissue, to determine bone density, to determine muscle tissue density, to determine water content, and so forth.

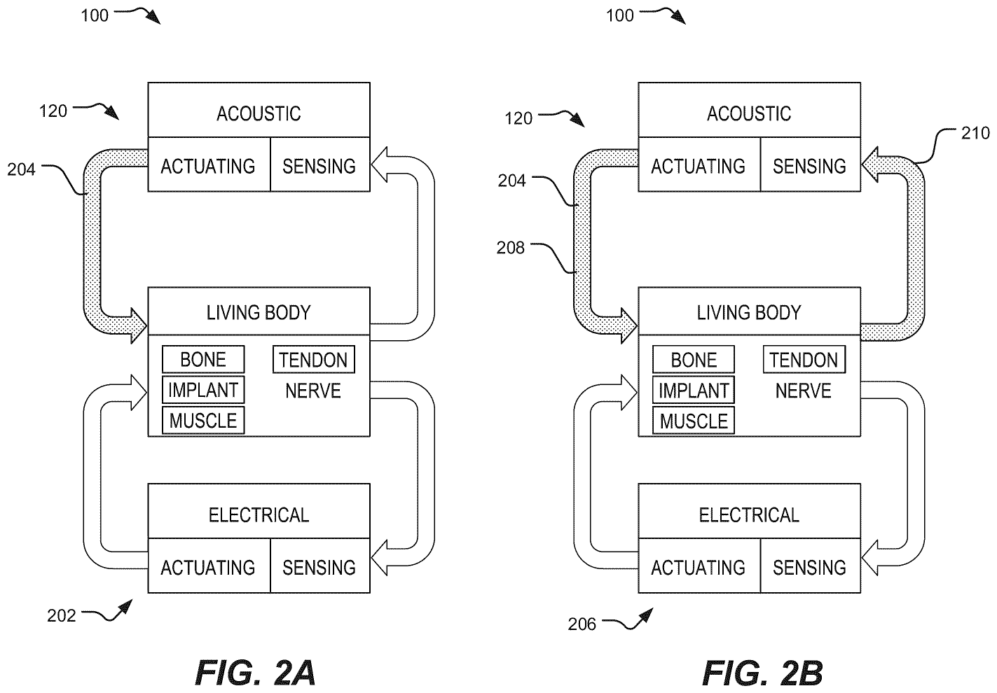

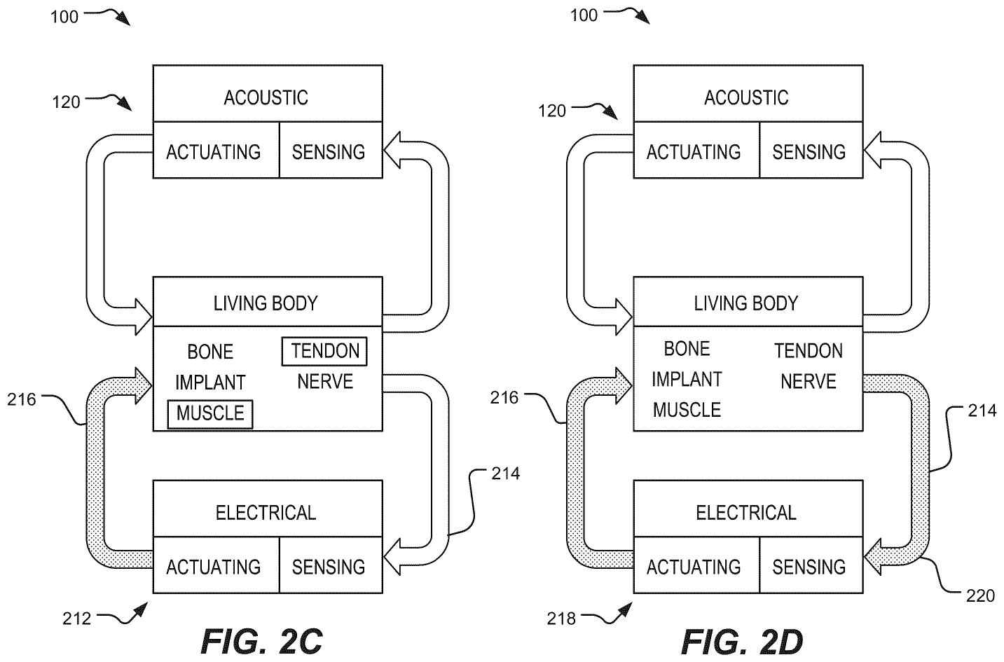

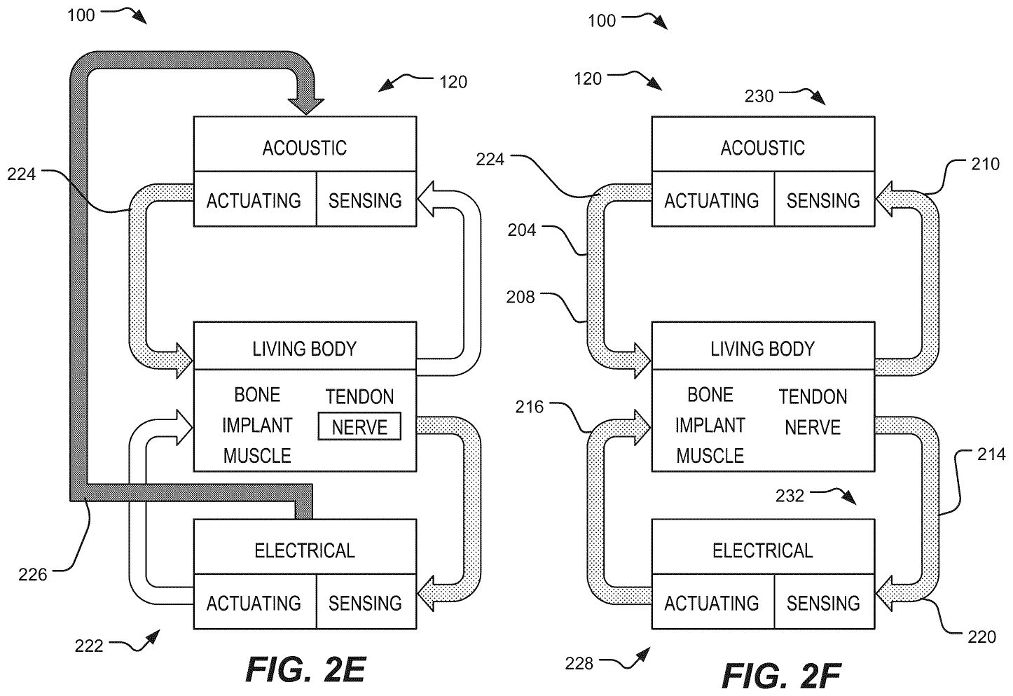

FIGS. 2A-2F depict diagrams of one or more cross-domain analyses 120 illustrating the cross domain or hybrid nature of using acoustic and electrical signals and sensing simultaneously. This not only means that acoustic and electrical stimulations and sensing can be performed at a same time and independently, but it also means that the system 100 can use acoustic stimulations to change electrical properties of tissue to inform the electrical sensing, or, as another example, electrical stimulation and/or sensing can inform acoustic stimulation or sensing.

FIG. 2A depicts an example acoustic therapeutic system 100 provided by the wearable device 102. The wearable device 102 can be operable to provide, in at least one configuration 202, only acoustic excitation 204. For example, in-phase excitation can be used to transmit acoustic energy to large areas. Furthermore, one or more metamaterials can be formed onto the acoustic transducers to form one various acoustic focusing, vortexing, and/or transmitting features. For example, any of the acoustic transducers or sensors can have a layer of metamaterial attached to its end to enhance or manipulate the sending and receiving of acoustic signals. The metamaterial can be a first type of material forming a spiral or gradient relative to a second material such that the metamaterial forms an acoustic lumbung lens, a vortexing lens, or so forth, to further focus the acoustic signal at a particular point or region of the target area. Moreover, phased array excitation can be used to focus acoustic energy at a small area. As an example, an array of acoustic transducers around an arm can stimulate a certain muscle or tendon for better healing or improvement.

FIG. 2B depicts an example acoustic diagnostics and monitoring system provided by the wearable device 102 in a second configuration 206. Static and dynamic measurement of geometrical and mechanical characteristics of different tissues, nerves, bones and implants can be generated. For instance, one or more actuators (e.g., acoustic actuators 107) can generate an acoustic signal 208. As the acoustic waves of the acoustic signal 208 propagate through at least a portion of the body, the acoustic waves can be modified based on geometry and mechanical properties of body parts. The modified acoustic waves 210 can then be sensed using an array of acoustic sensors 109 disposed on the wearable device 102.

FIG. 2C depicts an example electromyography (EMG) system provided by the wearable device 102. For instance, in at least a third configuration 212, electrical sensor 113 electrode only are used to sense electrical signals 214 transmitted by nerves or signals generated by muscles. The wearable device 102 can include a dense array 117 of electrodes to provide more accurate information of nerve and muscle health. Alternatively, the third configuration 212 can include only an electrical stimulation signal 216 sent to the living tissue from the electrical actuator 111

FIG. 2D depicts an example electrical capacitance/impedance measurement system provided by the wearable device 102 in a fourth configuration 218. For example, one or more electrodes can be used to generate and apply the electrical signals 216 (e.g., stimulation signals) with different frequencies and/or wave forms. The electrical signals 216 can propagate through different body parts, and the electrical signals 216 can be modified based on electrical capacitance and/or resistance of different body parts. The modified signal(s) 220 can be measured using an array of electrodes. As such, information relating to a special location including volumetric changes, body composition, electrical properties, and/or nerve activates can be extracted from the measured signal 214.

FIG. 2E depicts an example electro-acoustic stimulation system provided by the wearable device 102 in a fifth configuration 222 which can be used to stimulate nerves. For instance, acoustic actuators 109 disposed on the wearable device 102 can be used for neuromodulation 224 by focusing sound on a particular nerve. Acoustic sensors 111 disposed on the wearable device 102 can sense the acoustic field generated by the acoustic actuators 109 to determine the occurrence of acoustic stimulation. Moreover, electrodes can be used simultaneously to sense the nerve's electrical reaction 226 to acoustic stimulation.

FIG. 2F depicts an example electro-acoustic muscle characterization system provided by the wearable device 102 in a sixth configuration 228. For example, the wearable device 102 can provide acoustic and electrical monitoring systems that operate simultaneously. The acoustic monitoring system 230 can provide information about geometrical properties and/or other physical properties of the targeted body part. The electrical monitoring system 232 can provide information about nerves and muscle activities, geometrical and some physical characteristics of the body part. In some scenarios, the information of these two system can be fused together to provide an accurate understanding of the body part. For instance, it is to be understood that any of the configurations depicted in FIGS. 2A-2F (e.g., the first configuration 202, the second configuration 206, the third configuration 212, the fourth configuration 214, the fifth configuration 222, and/or the sixth configuration 21X) can be combined with any configuration, and any portion(s) of any configuration(s) can be combined with any other portion(s).

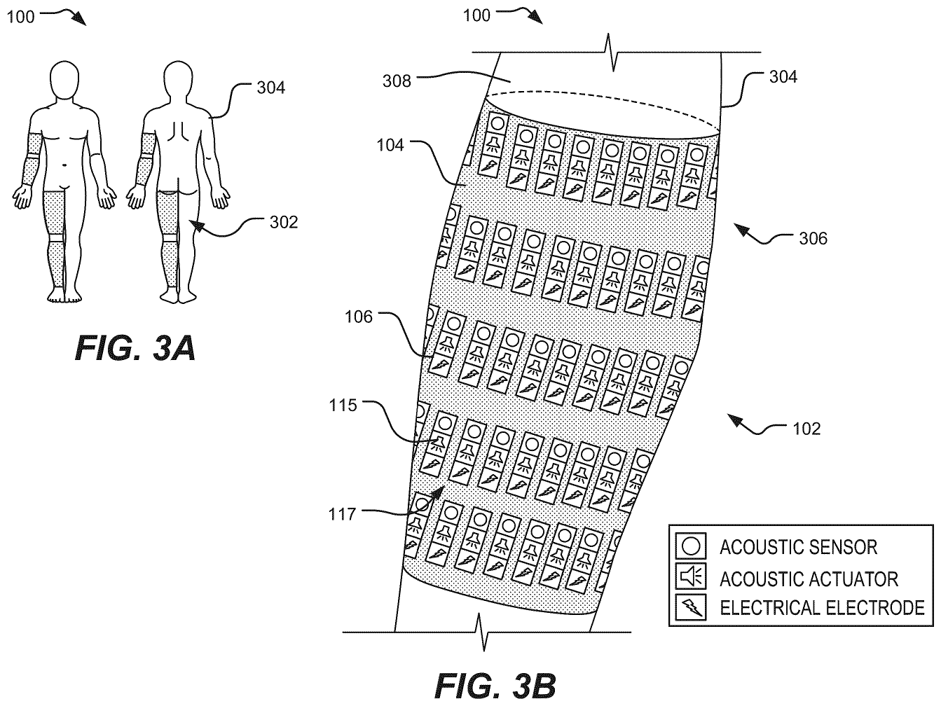

FIGS. 3A-6B illustrate example wearable devices 102 for targeting particular body parts 302 of a user 304. For instance, FIGS. 3A and 3B depict a wearable cuff or sleeve 306 that can be pulled over and/or can target an upper arm 308 (e.g., bicep and/or tricep), a lower arm (e.g., forearm), an upper leg (e.g., thigh), a lower leg (e.g., calf), and/or combinations thereof. The wearable device 102 can include an array of acoustic sensors 109, acoustic actuators 111, and electrical sensors 113 and/or electrical actuators 111 (e.g., the plurality of sensors/actuators 106) disposed at least partly or fully around the wearable device 102.

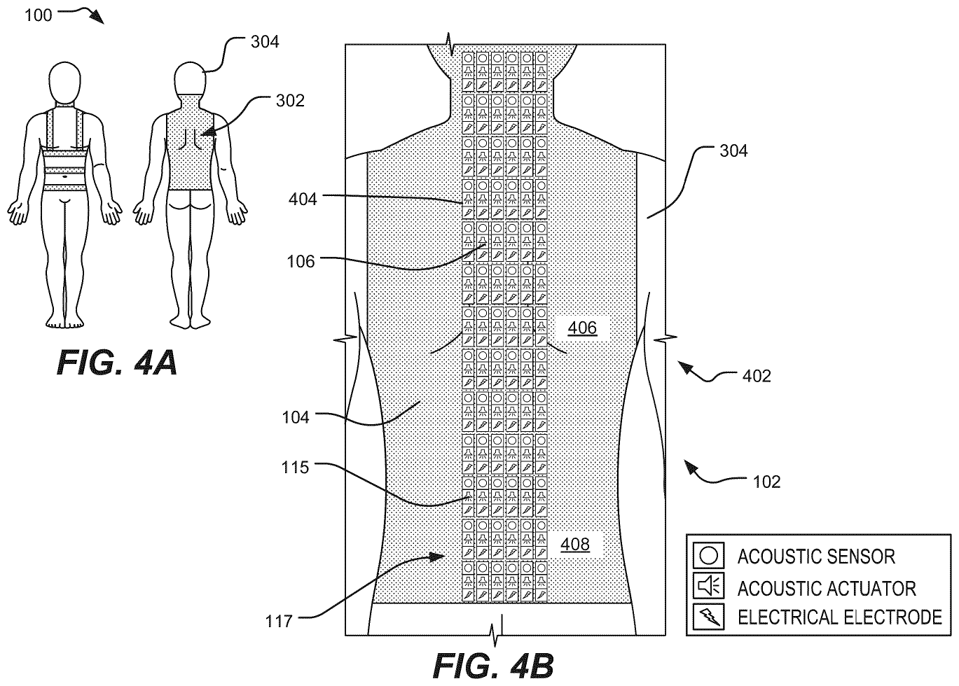

FIGS. 4A and 4B depict an example wearable back harness 402 and/or spinal strip for targeting a spine 404 of the user 304. For instance, the wearable device 102 can include a back portion 406 and/or one or more front portions (e.g., front straps) to maintain the wearable device 102 in place on a back of the user 304. The array 117 of sensors and/or actuators (e.g., acoustic sensors 109, acoustic actuators 107, electrical electrodes 111/113, and/or integrated sensors/actuators 115) can be disposed on a portion of the wearable device 102, such as a substantially elongated rectangular portion 408 covering a spinal region and/or rear neck region of the user 304.

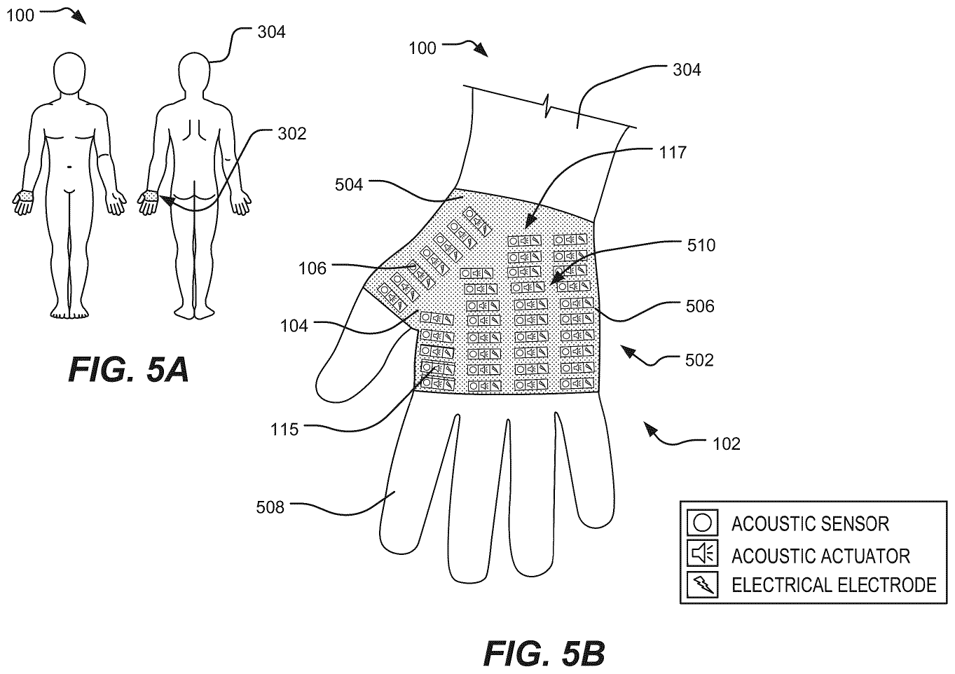

FIGS. 5A and 5B depict an example wearable glove 502 for targeting a hand region 504 of the user 304. For instance, the wearable glove 502 can include the array 117 of sensors and/or actuators disposed on a palm portion and/or a back-of-hand portion of the wearable glove 502. The wearable glove 502 can be a cut-off glove 506 omitting finger portions or, alternatively, can include one or more finger portion(s) having one or more sensors/actuators for targeting a finger region 508 of the user 304. Moreover, the wearable device 102 can be a wearable sock or slipper for targeting a foot region and/or toe region of the user 304. Some examples, of the wearable device 102 include a uniform or substantially uniform distribution 510 of the sensors/actuators 106 on the substrate material 104. Additionally or alternatively, the sensors/actuators 106 can have a localized or non-uniform distribution on the substrate material 104. For instance, the wearable glove 502 may have the acoustic actuators 107 disposed on a first side (e.g., a palm side) and the acoustic sensors 109 can be disposed on a second side (e.g., a back-of-hand side). The distribution of sensors/actuators 106 can correspond to a particular geometry of the tissue, implant, bone, or nerves being monitored and/or stimulated.

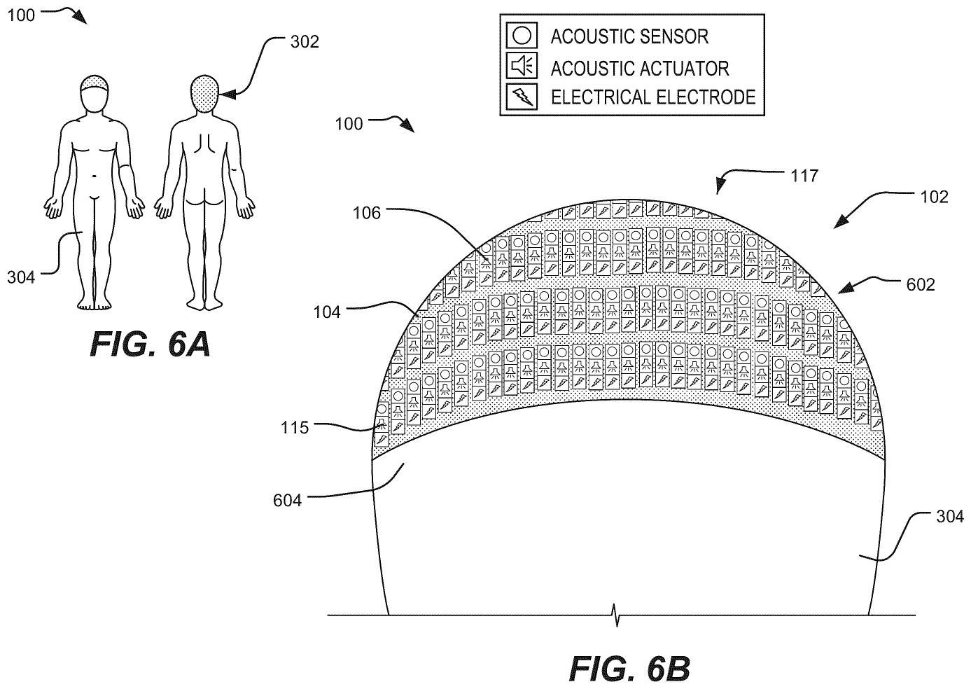

FIGS. 6A and 6B depict an example wearable cap 602 or head band for targeting a head region 604. The wearable device 102 can be operable to fit around a top portion of a user's head or cranium and/or can wrap around to cover a back of the user's head or cranium.

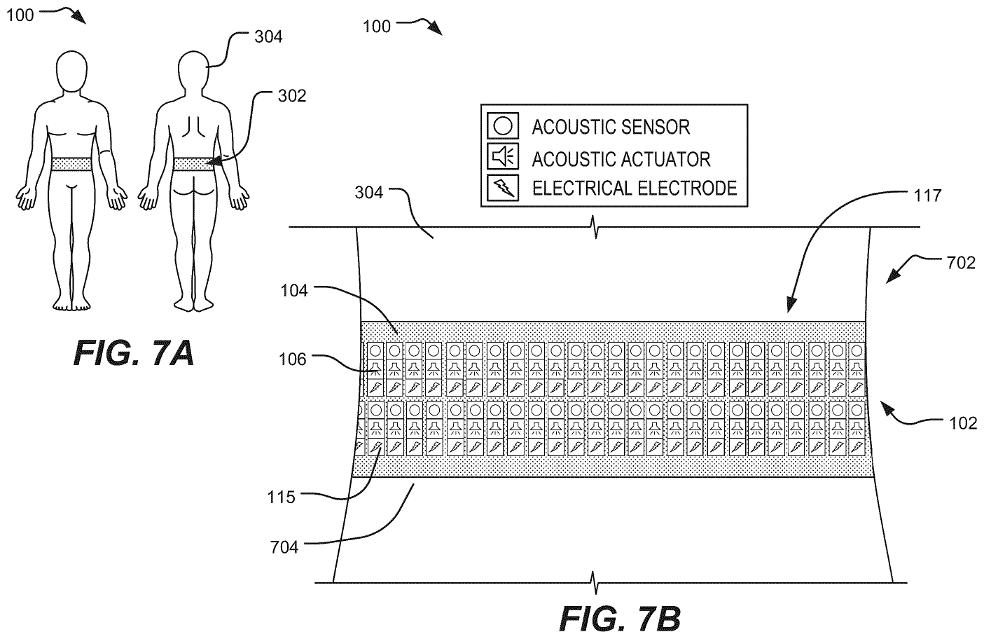

FIGS. 7A and 7B depict an example torso band 702 or waist band for targeting a torso region 704 of the user 304. The wearable device 102 can form a loop or band wrapped around the torso region 704, such as a strip with one or more securement mechanisms (e.g., hooks, buttons, hook-and-loops, e.g.,) to connect one end of the strip to another portion of the strip. Furthermore, the wearable device 102 can be a closed-loop band (e.g., a single circular piece) that is pulled over the legs of the user and up to the torso region. As discussed above, the wearable device 102 can be formed of a stretchy or flexible material to facilitate placement on and removal from the target region.

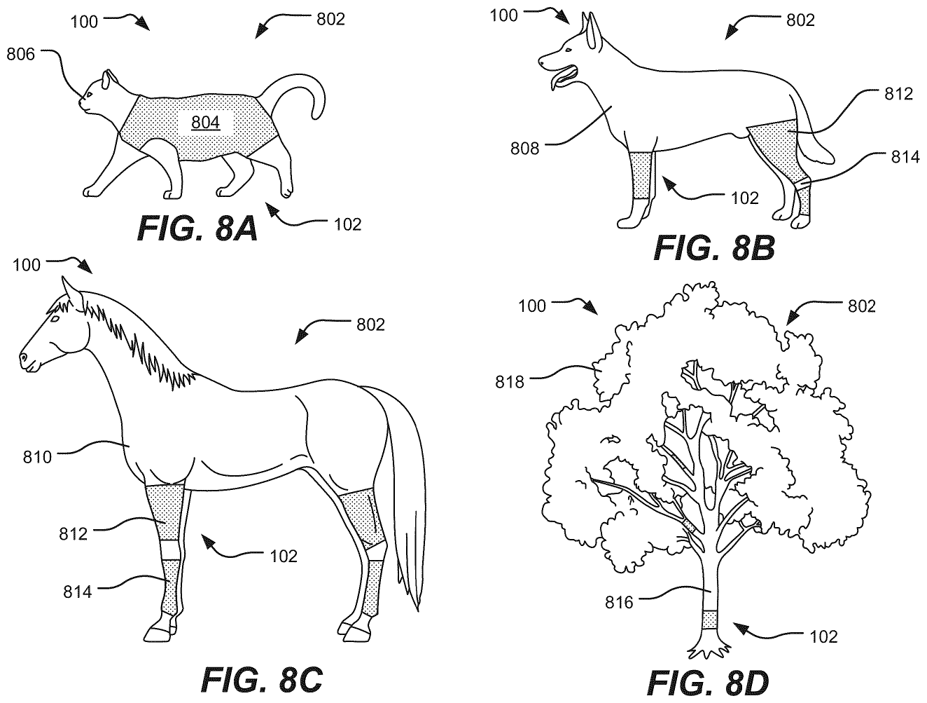

FIGS. 8A-8D depict example wearable devices 102 for use with non-human subjects 802. For instance, the wearable device 102 can be used as an animal husbandry device for sensing/actuating target regions of different animals or plants. The wearable device 102 can be a jacket or body sleeve 804, which can be disposed on a cat 806 (e.g., as shown in FIG. 8A), a dog 808 (e.g., as shown in FIG. 8B), and/or a horse 810 (e.g., as shown in FIG. 8C). Moreover, the wearable device 102 can form a strap, sleeve, or a tapered sleeve 812 for positioning over an upper portion or a lower portion of an animal leg 814. The wearable device 102 can also be a strap or sleeve for positioning over trunk 816 and/or branch of a plant or tree 818 (e.g., as shown in FIG. 8D).

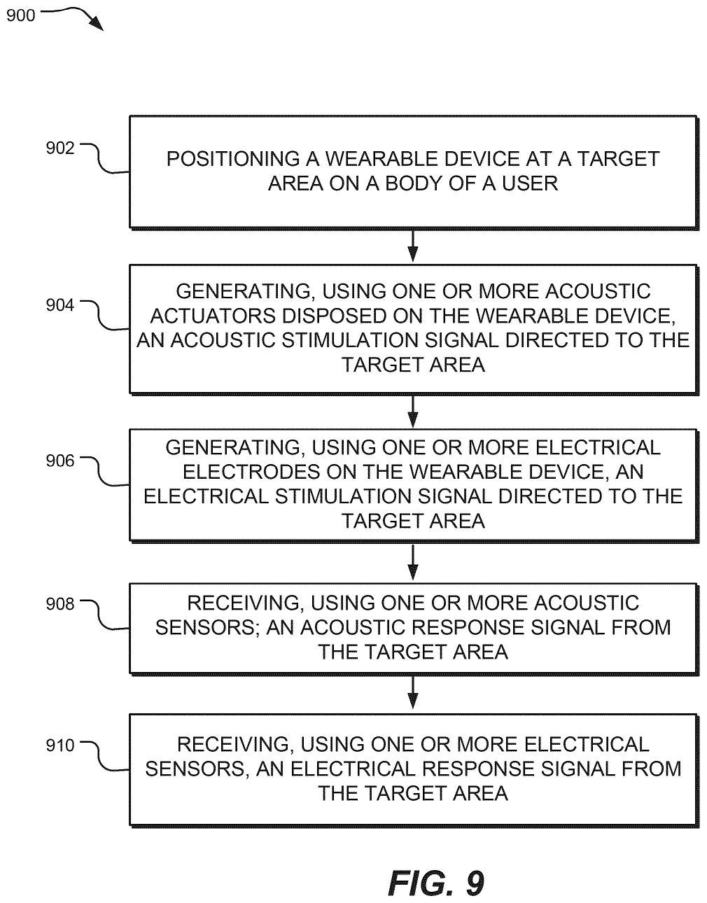

FIG. 9 depicts an example method 900 of performing tissue stimulation or analysis with a wearable device 102.

In some examples, at operation 902, the method 900 can position a wearable device at a target area on a body of a user. At operation 904, the method 900 can generate, using one or more acoustic actuators disposed on the wearable device, an acoustic stimulation signal directed to the target area. At operation 906, the method 900 can generate, using one or more electrical electrodes on the wearable device, an electrical stimulation signal directed to the target area. At operation 908, the method 900 can receive, using one or more acoustic sensors; an acoustic response signal from the target area. At operation 910, the method 900 can receive, using one or more electrical sensors, an electrical response signal from the target area.

It is to be understood that the specific order or hierarchy of steps in the method(s) depicted throughout this disclosure are instances of example approaches and can be rearranged while remaining within the disclosed subject matter. For instance, any of the operations depicted throughout this disclosure may be omitted, repeated, performed in parallel, performed in a different order, and/or combined with any other of the operations depicted throughout this disclosure.

While the present disclosure has been described with reference to various implementations, it will be understood that these implementations are illustrative and that the scope of the present disclosure is not limited to them. Many variations, modifications, additions, and improvements are possible. More generally, implementations in accordance with the present disclosure have been described in the context of particular implementations. Functionality may be separated or combined differently in various implementations of the disclosure or described with different terminology. These and other variations, modifications, additions, and improvements may fall within the scope of the disclosure as defined in the claims that follow.

What is claimed is:

1. A wearable device for stimulation or analysis of biological systems or implantable objects, the wearable device comprising:
a substrate material;
a plurality of acoustic actuators disposed on the substrate material and operable to generate an acoustic stimulation signal directed to a target area;
a plurality of acoustic sensors disposed on the substrate material and operable to receive an acoustic response signal from the target area; and
a plurality of electrical electrodes disposed on the substrate material and operable to generate an electrical stimulation signal directed to the target area and receive an electrical response from the target area.

2. The wearable device of claim 1,
wherein,
the substrate material includes a flexible materials including at least one of a fabric, a natural biological materials, a polymer, or a metal.

3. The wearable device of claim 1,
wherein,
the substrate material forms a sleeve or a cuff.

4. The wearable device of claim 3,
wherein,
the substrate material forms a glove.

5. The wearable device of claim 1,
wherein,
the substrate material forms a head cap.

6. The wearable device of claim 1,
wherein,
the substrate material forms a back harness, a waist binder, a torso binder, or an abdominal binder; and
the plurality of acoustic actuators, the plurality of acoustic sensors, and the plurality of electrical electrodes are positioned along a spinal region of the back harness, the waist binder, the torso binder, or the abdominal binder.

7. A wearable device for stimulation or analysis of biological systems or implantable objects, the wearable device comprising:
a substrate material; and
a plurality of sensors and actuators disposed on the substrate material as an array, the plurality of sensors and actuators are operable to:
generate an acoustic stimulation signal directed to a target area;
generate an electrical stimulation signal directed to the target area;
receive an acoustic response signal from the target area; and
receive an electrical response signal from the target area.

8. The wearable device of claim 7,
wherein,
the plurality of sensors and actuators are substantially evenly distributed on the substrate material.

9. The wearable device of claim 7,
wherein,
one of the plurality of sensors and actuators includes at least one of:
a piezoelectric transducer;
a speaker and a microphone; or
one or more metamaterials.

10. A method to stimulate or analyze biological systems or implantable objects, the method comprising:
positioning a wearable device at a target area on a body of a user;
generating, using one or more acoustic actuators disposed on the wearable device, an acoustic stimulation signal directed to the target area;
generating, using one or more electrical electrodes on the wearable device, an electrical stimulation signal directed to the target area;
receiving, using one or more acoustic sensors; an acoustic response signal from the target area; and
receiving, using one or more electrical sensors, an electrical response signal from the target area.

11. The method of claim 10, further comprising:
changing an electrical property of the target area with the acoustic stimulation signal, the electrical response signal representing the changing of the electrical property.

12. The method of claim 10, further comprising:
performing, with the acoustic stimulation signal, acoustic therapy or a quality assessment on at least one of:
muscle tissue,
bone tissue,
tendon tissue, or
implant tissue.

13. The method of claim 12, further comprising:
performing, with the acoustic stimulation signal or the acoustic response signal, acoustic monitoring for biological systems, or implantable objects.

14. The method of claim 12, further comprising:
performing, with the electrical response signal, an electromyography (EMG) for biological systems, muscle tissue, tendon tissue, or a nerve activate.

15. The method of claim 12, further comprising:
performing, with the electrical stimulation signal and the electrical response signal, an electrical impedance measurement or an electrical capacitance measurement for tissue of the target area.

16. The method of claim 15,
wherein,
the electrical impedance measurement or the electrical capacitance measurement indicates at least one of a volumetric change, a composition change, a health of a tissue or a biological system, a mechanical change, an electrical change, or a nerve activate.

17. The method of claim 15, further comprising:
performing, with the acoustic stimulation signal and the electrical response signal, an electro-acoustic stimulation in which the acoustic stimulation signal stimulates a nerve, and the nerve generates the electrical response signal.

18. The method of claim 12, further comprising:
performing, with the acoustic stimulation signal and the electrical response signal, an electro-acoustic muscle characterization in which the electrical response signal or the acoustic response signal is generated responsive to the acoustic stimulation signal and the electrical stimulation signal.

19. The method of claim 12,
wherein,
a frequency or an intensity of the acoustic stimulation signal corresponds to a type of tissue, organ, or cells at the target area.

20. The method of claim 19,
wherein,
the frequency is an ultrasonic frequency, and the type of tissue includes a tendon, a muscle, a bone, or cartilage receiving the acoustic stimulation signal for healing.
